(19) United States
(12) Patent Application Publication
(10) Pub. No.: US 2012/0156216 A1
(43) Pub. Date: Jun. 21, 2012

(54) Methods and Compositions for Treatment of Tumor Metastasis

(76) Inventor: Youngman OH, Glen Allen, VA (US)

(21) Appl. No.: 13/254,709

(22) PCT Filed: Mar. 2, 2010
(86) PCT No.: PCT/US2010/025879

§ 371 Date: Mar. 6, 2012

Related U.S. Application Data
(60) Provisional application No. 61/156,686, filed on Mar. 2, 2009.

(51) Int. Cl. A61K 39/395; A61K 31/366; C07D 309/30; A61K 31/22; A61P 35/04; C07K 16/18; A61K 31/59; A61K 31/375; G01N 33/574; C07K 16/22; C07C 69/013

(52) U.S. Cl. 424/158.1; 424/172.1; 530/389.2; 514/460; 549/292; 514/546; 560/256; 530/389.1; 514/167; 514/474; 436/501

(57) ABSTRACT

The various embodiments of the present invention are directed to methods and compositions for treatment, inhibition or reduction of metastasis in cancers. In particular, the invention is related to compositions and methods affecting connective tissue growth factor (CTGF). An aspect of the present invention comprises a method for interfering with the activity of connective tissue growth factor, comprising, administering to a subject an effective amount of a composition that inhibits production of or activity of connective tissue growth factor (CTGF), wherein the composition interferes with the activity of CTGF or prevents the transcription of CTGF genes or translation of CTGF mRNA. For example, the composition can comprises at least one HMG-CoA reductase inhibitor compound, such as a statin.

### BACKGROUND OF THE INVENTION

(1) Technical Field of the Invention

The present invention is related to methods and compositions for treatment or prevention of the metastasis of cancers. In particular, the invention is related to compositions and methods affecting the production and activity of connective tissue growth factor (CTGF).

(2) Description of Related Art

The insulin-like growth factor (IGF) signaling pathway plays a role in the development and/or progression of cancer. As components of the IGF signaling pathway, the IGF binding proteins (IGFBPs) can act as either positive or negative regulators of the IGF signaling pathway. Several low-affinity members of the IGFBP family have been identified based on their structural and functional relationship to the IGFBPs and have been designated as IGFBP-related proteins (IGFBP-rPs). IGFBP-rPs are cysteine-rich proteins that serve as mediators of various biological functions, such as growth regulation. IGFBP-rPs include mac25 (IGFBP-rP1), connective tissue growth factor (CTGF or IGFBP-rP2), nephroblastoma over expressed (NOVH or IGFBP-rP3), and CYR61 (IGFBP-rP4).

CTGF is an immediate early gene that is potently induced by a variety of growth factors, such as platelet-derived growth factor (PDGF), fibroblast growth factor (FGF), vascular endothelial growth factor (VEGF), and transforming growth factor beta (TGF-β). CTGF is expressed by several stromal cell types, including fibroblasts, endothelial cells, smooth muscle cells, myofibroblasts, and some epithelial cell types in diverse tissues. Consistent with its role in connective tissue biology, CTGF is a potent regulator of fibroblast proliferation, cell adhesion, and the stimulation of extracellular matrix production. Experimental evidence demonstrates that CTGF plays an important role in the pathogenesis of fibrotic disorders such as idiopathic pulmonary fibrosis, scleroderma, diabetic nephropathy, glomerulosclerosis, cirrhosis, and diabetic retinopathy. CTGF has been recognized as a major downstream mediator of the chronic fibrotic effects of TGF-β; however, recent data indicate that CTGF can be produced in a TGF-β-independent manner and can induce fibrosis in fibroblasts. Furthermore, blocking CTGF expression using specific siRNAs or neutralizing antibodies results in suppression of expression of fibrotic proteins, such as fibronectin and collagens, thereby inhibiting the fibrotic response in systemic sclerosis, liver fibrosis, and idiopathic pulmonary fibrosis. This evidence strongly suggests that CTGF induces fibrosis by itself and is indispensable for TGF-β-induced fibrosis; however, CTGF signaling pathways involved in those biological actions are not known.

Recently, circumstantial evidence has emerged suggesting that CTGF plays a role in tumorigenesis and progression of a variety of human cancers. Elevated CTGF levels have been detected in a number of cancers including pancreatic, breast, glioblastoma, and esophageal cancers, and hepatocellular carcinoma. An increase in CTGF was also reported to be associated with decreased survival of patients with breast cancer, glioblastoma, and adenocarcinoma of the esophagus. In particular, significant correlations were found between CTGF expression and tumor stage, tumor size, lymph node status, and age at diagnosis in breast cancer, demonstrating the clinical significance of CTGF in the progression of breast cancer. These studies indicate that CTGF may play a critical role for tumorigenesis and progression of human cancer. Indeed, recent studies demonstrate that treatment with recombinant CTGF resulted in an increase of tumor growth, angiogenesis, and metastasis in breast and pancreatic cancer cells in vitro and in vivo. These studies suggest that CTGF may be a new molecular target for metastasis of human cancer.

Accordingly, there is a need for methods and compositions for inhibiting the activity of CTGF, particularly as related to neoplastic disease. There is also a need for systems and methods of diagnosing CTGF-related disorders. It is to the provision of such methods and compositions related to CTGF that the various embodiments of the present invention are directed.

### BRIEF SUMMARY OF THE INVENTION

Various embodiments of the present invention are directed to methods and compositions for treatment, inhibition, prevention, or reduction of metastasis in cancers. In particular, the invention is related to compositions and methods affecting the production and activity of connective tissue growth factor (CTGF).

An aspect of the present invention comprises a method for interfering with the activity of connective tissue growth factor (CTGF), comprising, administering to a subject an effective amount of a composition comprising an inhibitor of CTGF. In an embodiment of the present invention, the composition prevents the transcription of CTGF genes or translation of CTGF mRNA. In another embodiment of the present invention, the composition interferes with the activity of CTGF cellular signaling pathways. The composition that interferes with the activity can comprise an antibody or a fragment thereof that binds to at least a portion of CTGF, a peptide, a nucleic acid, or small molecule. More particularly, the composition can comprise at least one HMG-CoA reductase inhibitor compound. The at least one HMG-CoA reductase inhibitor compound can be selected from atorvastatin, berivastatin, cerivastatin, compactin, dalvastatin, dihydromevinolin, fluvastatin, glenvastatin, lovastatin, mevastatin, nisvastatin, pravastatin, pitavastatin, rivastatin, rosuvastatin, simvastatin, visastatin, or combinations thereof. In an exemplary embodiment of the present invention, the HMG-CoA reductase inhibitor compound is simvastatin. In addition, the composition can further comprises at least one of Vitamin D or derivatives thereof, Vitamin C or derivatives thereof, capsaicin or derivatives thereof, inhibitors of cathepsins, caspases, or combinations thereof.

In an embodiment of the present invention, a method for interfering with the activity of connective tissue growth factor (CTGF) can comprise interfering with the activity of CTGF associated with cancer metastasis. In another embodiment of the present invention, a method for interfering with the activity of connective tissue growth factor (CTGF) can comprise interfering with the activity of CTGF associated with prostate cancer metastasis. In yet an embodiment of the present invention, a method for interfering with the activity of connective tissue growth factor (CTGF) can comprise interfering with the activity of CTGF associated with cancer metastasis. In still another embodiment of the present invention, a method for interfering with the activity of connective tissue growth factor (CTGF) can comprise interfering with the activity of CTGF associated with breast cancer metastasis.

Another aspect of the present invention comprises a pharmaceutical composition comprising an inhibitor of connective tissue growth factor (CTGF). In an embodiment of the present invention, the inhibitor of CTGF can compromise an antibody or a fragment thereof that binds to at least a portion of CTGF, a peptide, a nucleic acid, or small molecule and is capable of at least one of interfering with the activity of CTGF; preventing the transcription of CTGF genes; or translation of CTGF mRNA. More particularly, the inhibitor of CTGF can comprise at least one HMG-CoA reductase inhibitor compound. The at least one HMG-CoA reductase inhibitor compound is selected from atorvastatin, berivastatin, cerivastatin, compactin, dalvastatin, dihydromevinolin, fluvastatin, glenvastatin, lovastatin, mevastatin, nisvastatin, pravastatin, pitavastatin, rivastatin, rosuvastatin, simvastatin, visastatin or combinations thereof. In addition, the composition can further comprise at least one of Vitamin D or derivatives thereof, Vitamin C or derivatives thereof, capsaicin or derivatives thereof, inhibitors of cathepsins, caspases, or combinations thereof.

Another aspect of the present invention is a system for diagnosing the progression of cancer in a subject, comprising a probe capable of detecting the expression of connective tissue growth factor (CTGF) in a subject. In one embodiment of the present invention, detecting the expression of CTGF in a subject comprises detecting expression of CTGF mRNA in a subject. In another embodiment of the present invention, detecting the expression of CTGF mRNA in a subject comprises detecting expression of CTGF mRNA in a tumor cell or a mesenchymal cell. In yet another embodiment of the present invention, detecting the expression of CTGF in a subject comprises detecting expression of CTGF protein in a subject. In still another embodiment of the present invention, detecting the expression of CTGF protein in a subject comprises detecting expression of CTGF protein in a tumor cell or a mesenchymal cell. In many of these embodiments the probe can comprises a nucleic acid or a protein. In an embodiment of the present invention, the system can further comprise a detector capable of detecting the interaction of the probe with a target associated with CTGF expression. In an embodiment of the present invention, CTGF is expressed in non-metastatic cells at a first amount and CTGF is expressed in metastatic cells at a second amount that is greater than the first amount.

Another aspect of the present invention comprises a method for diagnosing the progression of cancer in a subject, comprising: detecting the expression of connective tissue growth factor (CTGF) in a subject; and diagnosing the progression of cancer in a subject. In an embodiment of the present invention, detecting the expression of CTGF in a subject comprises detecting expression of CTGF mRNA in a subject. In another embodiment of the present invention, detecting the expression of CTGF mRNA in a subject comprises detecting expression of CTGF mRNA in a tumor cell or a mesenchymal cell. In yet another embodiment of the present invention, detecting the expression of CTGF in a subject comprises detecting expression of CTGF protein in a subject. In yet another embodiment of the present invention, detecting the expression of CTGF protein in a subject comprises detecting expression of CTGF protein in a tumor cell or a mesenchymal cell. In an embodiment of the present invention, diagnosing the progression of cancer in a subject comprises determining the expression of CTGF expression in a subject, wherein CTGF is expressed in non-metastatic state at a first amount and CTGF is expressed in a metastatic state at a second amount that is greater than the first amount.

Another aspect of the present invention comprises a method for treating a neoplastic disease, comprising, administering to a human an effective amount of a composition comprising a statin that inhibits production or activity of connective tissue growth factor (CTGF). In an embodiment of the present invention, the statin can be selected from one or more of atorvastatin, berivastatin, cerivastatin, compactin, dalvastatin, dihydromevinolin, fluvastatin, glenvastatin, lovastatin, mevastatin, nisvastatin, pravastatin, pitavastatin, rivastatin, rosuvastatin, simvastatin, visastatin, or combinations thereof. In an exemplary embodiment of the present invention, the statin is simvastatin. In addition, the composition can further comprise at least one of Vitamin D or derivatives thereof, Vitamin C or derivatives thereof, capsaicin or derivatives thereof, inhibitors of cathepsins, caspases, or combinations thereof. In an embodiment of the present invention, method for treating a neoplastic disease can further comprising reducing the metastasis of a cancer in a human. In one embodiment of the present invention, the cancer is prostate cancer. In another embodiment of the present invention, the cancer is breast cancer.

Another aspect of the present invention includes a method for treating a radiation-induced cell growth comprising, administering to a human an effective amount of a composition comprising a statin that inhibits production or activity of connective tissue growth factor (CTGF). For example, the statin can be selected from one or more of atorvastatin, berivastatin, cerivastatin, compactin, dalvastatin, dihydromevinolin, fluvastatin, glenvastatin, lovastatin, mevastatin, nisvastatin, pravastatin, pitavastatin, rivastatin, rosuvastatin, simvastatin, visastatin, or combinations thereof. In an exemplary embodiment of the method, the statin is simvastatin. The composition can further comprises at least one of Vitamin D or derivatives thereof, Vitamin C or derivatives thereof, capsaicin or derivatives thereof, inhibitors of cathepsins, caspases, or combinations thereof. The method for treating a radiation-induced cell growth can further comprising at least one of reducing the metastasis of a cancer in a human, reducing the invasiveness of a cancer in a human, or reducing the angiogenesis of a cancer in a human, such as for example, prostate cancer or breast cancer.

Other aspects and features of embodiments of the present invention will become apparent to those of ordinary skill in the art, upon reviewing the following description of specific, exemplary embodiments of the present invention in conjunction with the accompanying figures.

### BRIEF DESCRIPTION OF DRAWINGS

FIG. 1 illustrates the expression profile of CTGF in prostate cancer cells.

FIG. 2 demonstrates overexpression of CTGF and regulation of EMT markers.

FIGS. 3 A-B shows the suppression of CTGF production by statins.

FIGS. 4 A-B illustrate the inhibitory effect of simvastatin on TGF-β1-induced CTGF and fibronectin expression in stromal fibroblasts.

FIGS. 5 A-D demonstrate the effect of CTGF on EMT marker expression at mRNA levels and protein levels in M12 and M2182 prostate cancer cells.

FIGS. 6 A-D show the inhibitory effect of simvastatin on CTGF-induced regulation of EMT marker expression at mRNA levels and protein levels in M12 and M2182 prostate cancer cells.

FIGS. 7 A-B demonstrate the effect of CTGF on EMT marker expression and the inhibitory effect of simvastatin on CTGF-induced regulation of EMT marker expression in MCF-7 breast cancer cells.

FIGS. 8 A-B demonstrate radiation-induced CTGF expression in M12 prostate cancer cells (FIG. 8A) and BEAS-2B-CSC lung cancer cells (FIG. 8B).

FIGS. 9 A-B demonstrate the effect of statins on radiation-induced CTGF expression in M12 prostate cancer cells (FIG. 9A) and BEAS-2B-CSC lung cancer cells (FIG. 9B).

### DETAILED DESCRIPTION OF THE INVENTION

Various embodiments of the present invention comprise methods and compositions for the treatment, prevention, or amelioration of disease by inhibiting CTGF. More specifically, the various embodiments of the present invention comprise methods and compositions for the treatment, prevention, or reduction of cancer metastasis by interfering with production of CTGF or the activity of CTGF. The various embodiments of the present invention comprise methods and compositions for the treatment, prevention, or reduction of the production of CTGF or the activity of CTGF in normal cells and tissues including, but not limited to, fibroblasts, endothelial cells and muscle cells, and tumor cells or tissues.

CTGF plays a role in tumorigenesis and progression of a variety of human cancers. Elevated CTGF levels have been detected in a number of cancers including pancreatic, breast, glioblastoma, and esophageal cancers, and hepatocellular carcinoma. An increase in CTGF was also reported to be associated with decreased survival of patients with breast cancer, glioblastoma, and adenocarcinoma of the esophagus. In particular, significant correlations were found between CTGF expression and tumor stage, tumor size, lymph node status, and age at diagnosis in breast cancer, demonstrating clinical significance of CTGF in the progression of breast cancer. These studies indicate that CTGF may play a critical role for tumorigenesis and progression of human cancer. Indeed, recent studies demonstrate that treatment with recombinant CTGF resulted in an increase of tumor growth, angiogenesis, and metastasis in breast and pancreatic cancer cells in vitro and in vivo.

Despite this circumstantial data, the etiology and cellular factors associated with cancer metastasis are unclear, prior to the present invention. For example, a CTGF-specific monoclonal antibody has been produced, and subsequent use of this CTGF monoclonal antibody in antibody therapy resulted in significant attenuation of tumor growth, microvasculature, metastasis in orthotopic mouse models of breast and pancreatic cancer. These findings suggest that CTGF may contribute to aberrant autocrine/paracrine pathways that promote cancer cell growth, invasion, and metastasis. Therefore, blocking CTGF actions may represent a novel therapeutic approach in human cancer, which exhibit elevated expression of CTGF. However, the underlying mechanisms involved in tumor growth and metastatic effects of CTGF have not been elucidated.

In the case of prostate cancer, only a few studies have been reported with respect to biological actions of CTGF. Elevated expression of CTGF has been reported in prostate cancer, and it appears that CTGF expression was observed more strongly in epithelial cells than in the stromal area in prostate tissue. Further studies have demonstrated that CTGF was expressed at low levels in non-tumor-promoting prostate stromal cells, and CTGF was constitutively expressed in tumor-promoting prostate stromal cells.

Similar to other cancers, TGF-β is a potent inducer of CTGF in prostate cancer cells as well as prostate stroma. Furthermore, when mouse prostate stromal fibroblasts expressing retroviral-introduced CTGF were combined with LNCaP human prostate cancer cells in the DRS xenograft tumor model, stromal expression of CTGF resulted in an increase of angiogenesis and tumorigenesis of prostate cancer. Taken together, these studies indicate that CTGF is a downstream mediator of TGF-β action in cancer cells and cancer-associated reactive stroma.

TGF-β is overexpressed by cancer epithelial cells in most carcinomas, including prostate cancer. The ability of TGF-β to enhance tumorigenicity in vivo results from its role in many key processes, including stimulating angiogenesis, inhibiting immune surveillance, or promoting the degradation of the extracellular matrix. TGF-β can also promote local invasion and metastasis through the epithelial to mesenchymal transition (EMT). The conversion of an epithelial cell into a mesenchymal cell requires alterations in morphology, cellular architecture, adhesion, and migration.

There is mounting evidence that the acquisition of migratory and invasive properties by epithelial cells occurs in response to the microenvironment, which is associated with gain of mesenchymal characteristics and loss of epithelial characteristics. For instance, cells in the center of a malignant prostate tumor maintain an epithelial phenotype, whereas cells at the invasive front of a malignant prostate tumor have a mesenchymal phenotype, characterized by increased expression of mesenchymal markers, such as N-cadherin and vimentin. Recent studies indicate that a variety of growth factors, such as TGF-β, VEGF and PDGF, and transcriptional factors, including hypoxia-inducible factor 1α(HIF-1α), promote the invasion potency of prostate cancer cells through EMT induction, as indicated by loss of E-cadherin and cytokeratins and gain in expression of N-cadherin, vimentin and fibronectin. Interestingly, these EMT-inducing factors, TGF-β, VEGF, PDGF, and HIF-1α, have also been demonstrated to stimulate CTGF expression in prostate cancer cells, fibroblasts, and endothelial cells. Taken together, these studies provide circumstantial evidence that CTGF may promote prostate cancer cell invasion and metastasis via induction of EMT, as cooperating with, or being a downstream mediator of these EMT-inducing growth factors, thereby participating in aberrant autocrine and paracrine pathways in prostate cancer that contribute to altered stromal-epithelial interactions and that promote prostate cancer cell invasion and metastasis.

In view of the involvement of the microenvironment in cancer metastasis, CTGF is an excellent molecular target for metastasis of human cancer (e.g., breast, colon, lung, kidney, liver, melanoma, rectal, and/or prostate cancer). An aspect of the present invention comprises methods for interfering with the activity of CTGF comprising, administering to a subject an effective amount of a composition that inhibits, reduces, prevents, or alters production of or interferes with the activity of connective tissue growth factor (CTGF).

The term “composition” as used herein can refer to one or more compounds or substances, such as a pharmaceutical compound, a therapeutic compound, an active agent, or the like. Thus, the term “composition” and the term “compound” may be used interchangeably throughout this specification as a composition can comprise a compound. In addition, the term “composition” can refer to more than compounds or substances. A composition may also comprise one or more pharmaceutical additives including, but not limited to, solubilizers, emulsifiers, buffers, preservatives, suspending agents, thickening agents, stabilizers, inert components, and the like.

As used herein, the phrase “interfering with the activity of CTGF” can refer to both direct and indirect interference with the activity of CTGF protein, and direct or indirect interference with the transcription of CTGF genes or the translation of CTGF mRNA. Furthermore, “interfering with the activity of CTGF” can include partially interfering with the activity of CTGF, substantially interfering with the activity of CTGF, or completely interfering with the activity of CTGF.

As used herein, the term “subject” encompasses humans and other animals, for example a mammal (e.g., a cow, a horse, a dog, a cat, a goat, a sheep, a primate, a mouse, a rabbit, a pig, a rat, a mouse, a guinea pig), a bird, a fish, an amphibian, or an insect.

Another aspect of the present invention comprises methods for treating cancer metastasis the method comprising administering to a subject an effective amount of a composition that inhibits, reduces, prevents, or alters production of or interferes with the activity of connective tissue growth factor (CTGF); and thus preventing, reducing, or inhibiting cancer metastasis, cancer cell growth, cancer invasion, and/or cancer angiogenesis. As used herein, the terms “preventing,” “reducing,” “altering,” or “inhibiting” refer to a difference in degree from a first state, such as an untreated state in a subject, to a second state, such as a treated state in a subject. For example, in the absence of treatment with the methods or compositions of the present invention, cancer metastasis may occur at a first metastatic rate. If a subject is exposed to treatment with the methods or compositions of the present invention, cancer metastasis occurs at a second metastatic rate that is altered, lessened, or reduced from the first metastatic rate. In another example, in the absence of treatment with the methods or compositions of the present invention, CTGF may be produced or expressed at a first level; however, if exposed to treatment with the methods or compositions of the present invention, CTGF production or expression is altered, lessened, inhibited, or reduced from the first level. The terms “preventing,” “reducing,” “altering,” or “inhibiting” may be used interchangeably through this application and may refer to a partial reduction, substantial reduction, near-complete reduction, complete reduction, or absence of cancer metastasis and the rate thereof.

Methods of the present invention comprise administering compounds or compositions to inhibit, reduce, prevent, or alter expression or production or interfere with the activity of CTGF activity in a subject. An aspect of the present invention comprises a pharmaceutical composition comprising an inhibitor of CTGF. As used herein, the phrases “inhibitor of CTGF” or “inhibits the production or expression of CTFF” refers to stopping, inhibiting, preventing, reducing, lessening, or altering expression or activity of CTGF in a subject, which may include interfering with effective action of CTGF in cellular pathways in which CTGF is active, for example as a signaling factor, or interfering with the transcription of CTGF genes or the translation of CTGF mRNA, among others.

Methods for interfering with the activity of CTGF or treating cancer metastasis comprise administration of a compound or composition comprising inhibitors of CTGF including, but not limited to, an antibody or fragment thereof capable of binding to at least a portion of CTGF, a small molecule, an interfering peptide that interferes with the activity of CTGF or a cellular factor related thereto, or an interfering nucleic acid (e.g., siRNA, antisense RNA) that interferes with the transcription of CTGF genes or genes related to the transcription of CTGF genes, or interferes with the translation of CTGF RNA or the RNAs of cellular factors that affect the expression or activity of CTGF.

Embodiments of the present invention contemplate the use of antibodies for inhibition of CTGF for use in the treatment methods and compositions disclosed herein. As used herein, the term “antibody” includes intact monoclonal and polyclonal antibody molecules, as well as antibody fragments (such as, for example, Fab and F(ab′)2 fragments). Fab and F(ab′)2 fragments lack the Fc fragment of an intact antibody, clear more rapidly from the circulation, and may have less non-specific tissue binding of an intact antibody. Antibodies of the present invention may be humanized or not, and may have functional groups or tags associated with them for monitoring functions or for providing additional activities or functionalities.

Interfering nucleic acids (e.g., an oligonucleotide) can comprise nucleic acids that can specifically bind to a complementary nucleic acid sequence. By binding to an appropriate target sequence, an RNA-RNA, a DNA-DNA, or RNA-DNA duplex is formed. These nucleic acids are often termed “antisense” because they are complementary to the sense or coding strand of the gene. The oligonucleotide may also form a triple helix if bound to a DNA duplex. By binding to the target nucleic acid, oligonucleotides can inhibit the function of the target nucleic acid. This could, for example, be a result of blocking the transcription, processing, poly(A) addition, replication, translation, or promoting inhibitory mechanisms of CTGF, such as promoting RNA degradation.

Interfering nucleic acids can be prepared in the laboratory using standard laboratory protocols, as are known to those skilled in the art. The interfering nucleic acids may then be provided to a subject by, for example, injection, topical, mucosal application, aerosol, or nasal, enteral, ophthalmic, oral, parenteral, transdermal airway delivery. Interfering nucleic acids may be 15 to 35 bases in length; however, it is appreciated that it may be desirable to use oligonucleotides with lengths outside this range, for example 10, 11, 12, 13, or 14 bases, or 36, 37, 38, 39 or 40 bases, among others. The design of interfering nucleic acids is routine and can readily be performed by the skilled person. By “antisense” it is intended to include all methods of DNA and/or RNA interference, which are regarded for the purposes of this invention as a type of antisense technology.

An aspect of the present invention comprises a pharmaceutical composition comprising an inhibitor of CTGF. In an exemplary embodiment of the present invention, a composition comprises an HMG-CoA reductase inhibitor, which may also be referred to as a statin. As used herein, a “statin” or “HMG-CoA reductase inhibitor” include, but are not limited to, atorvastatin, berivastatin, BMS-180431, cerivastatin, compactin, CP-83101, dalvastatin, dihydromevinolin, fluvastatin, glenvastatin, L-669262, lovastatin, mevastatin, nisvastatin, pravastatin, pitavastatin, rivastatin, rosuvastatin, RP 61969, SDZ-265859, simvastatin, visastatin, or combinations thereof. Statins or HMG-CoA reductase inhibitors can have hypolipidemic properties as they are small-molecule inhibitors of 3-hydroxy-3-methylglutaryl coenzyme-A (HMG-CoA) reductase, preventing the conversion of 3-hydroxy-3-methylglutary-1-CoA to mevalonate, which is the rate-limiting step in cholesterol synthesis.

The chemical names of some exemplary HMG-CoA reductase inhibitors are as follows: atorvastatin (((βR,δR)-2-(4-fluorophenyl)-β,δ-dihydroxy-5-(1-methylethyl)-3-phenyl-4-[(phenylamino)carbonyl]-1H-Pyrrole-1-heptanoic acid); berivastatin ((R*,S*-(E)-7-(4-(4-fluorophenyl)spiro(2H-1-benzopyran-2,1′-cyclopentan)-3-yl)-3,5-dihydroxy-ethyl ester); BMS-180431 ((3R,5S,6E)-rel-9,9-bis(4-fluorophenyl)-3,5-dihydroxy-8-(1-methyl-1H-tetrazol-5-yl)-6,8-Nonadienoic acid); cerivastatin ((3R,5S,6E)-7-[4-(4-fluorophenyl)-5-(methoxymethyl)-2,6-bis(1-methylethyl)-3-pyridinyl]-3,5-dihydroxy-6-heptenoic acid); compactin ((2S)-2-methyl butanoic acid (1S,7S,8S,8aR)-1,2,3,7,8,8a-hexahydro-7-methy-8-[2-[(2R,4R)-tetrahydro-4-hydroxy-6-oxo-2H-pyran-2-yl]ethyl]-1-naphthalenyl ester); CP-83101 ((3R,5 S,6E)-rel-3,5-dihydroxy-9,9-diphenyl-6,8-Nonadienoic acid methyl ester); dalvastatin ((4R,6S)-rel-6-[(1E)-2-[2-(4-fluoro-3-methylphenyl)-4,4,6,6-tetramethyl-1-cyclohexen-1-yl]ethenyl]tetrahydro-4-hydroxy-2H-Pyran-2-one); dihydromevinolin ((2S)-2-methyl-butanoic acid (1S,3S,4aR,7S,8S,8aS)-1,2,3,4,4a,7,8,8a-octahydro-3,7-dimethyl-8-[2-[(2R,-4R)-tetrahydro-4-hydroxy-6-oxo-2H-pyran-2-yl]ethyl]-1-naphthalenyl ester); fluvastatin ((3R,5S,6E)-rel-7-[3-(4-fluorophenyl)-1-(1-methylethyl)-1H-indol-2-yl]-3,-5-dihydroxy-6-heptenoic acid); glenvastatin ((4R,6S)-6-[(1E)-2-[4-(4-fluorophenyl)-2-(1-methylethyl)-6-phenyl-3-pyridinyl]ethenyl]tetrahydro-4-hydroxy-2H-Pyran-2-one); L-669262 (2,2-dimethyl-butanoic acid (1S,7R,8R,8aR)-1,2,6,7,8,8a-hexahydro-3,7-dimethyl-6-oxo-8-[2-[(2R,4R)-tetrahydro-4-hydroxy-6-oxo-2H-pyran-2-yl]ethyl]-1-naphthalenyl ester); lovastatin ((2(S)-2-methyl-butanoic acid (1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthalenyl ester); mevastatin ((1S,7S,8S,8aR)-8-{2-[(2R,4R)-4-hydroxy-6-oxotetrahydro-2H-pyran-2-yl]ethyl}-7-methyl-1,2,3,7,8,8a-hexahydronaphthalen-1-yl (2S)-2-methylbutanoate); pravastatin(βR,δR,1S,2S,6S,8S,8aR)-1,2,6,7,8,8a-hexahydro-β,β,6-trihydroxy-2-methy-1-8-[(2S)-2-methyl-1-oxobutoxy]-1-naphthaleneheptanoic acid); pitavastatin ((3R,5S,6E)-7-[2-cyclopropyl-4-(4-fluorophenyl)-3-quinolinyl]-3,5-dihydroxy-6-heptenoic acid); rivastatin (sodium (3R,5S)-7-[4-(4-fluorophenyl)-5-(methoxymethyl)-2,6-dipropan-2-yl-pyridin-3-yl]-3,5-dihydroxy-hept-6-enoate); rosuvastatin ((3R,5S,6E)-7-[4-(4-fluorophenyl)-6-(1-methylethyl)-2-[methyl(methylsulfonyl)amino]-5-pyrimidinyl]-3,5-dihydroxy-6-heptenoic acid); RP 61969 ([2S-[2a(E),4β]]-;4-(4-fluorophenyl)-2-(1-methylethyl)-3-[2-(tetrahydro-4-hydroxy-6-oxo-2H-pyran-2-yl)ethenyl]-1(2H)-isoquinolinone); and simvastatin (2,2-dimethyl-butanoic acid (1S,3R,7S,8S,8aR)-1,2,3,7,8,8a-hexahydro-3,7-dimethyl-8-[2-[(2R,4R)-tetrahydro-4-hydroxy-6-oxo-2H-pyran-2-yl]ethyl]-1-naphthalenyl ester),

The chemical structures of some of these exemplary HMG-CoA reductase inhibitors are set forth below:

| Name | Chemical Name | Structure |
|---|---|---|
| Atorvastatin | (βR,δR)-2-(4- fluorophenyl)β,δ- dihydroxy-5-(1- methylethyl)-3-phenyl- 4 (phenylamino) carbonyl]-1H-Pyrrole- 1-heptanoic acid | 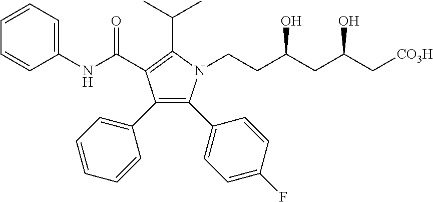 |
| Cerivastatin | (3R,5S,6E)-7-[4-(4- fluorophenyl)-5- (methoxymethyl)-2,6- bis(1-methylethyl)-3- pyridinyl]-3,5- dihydroxy-6-Heptenoic acid | 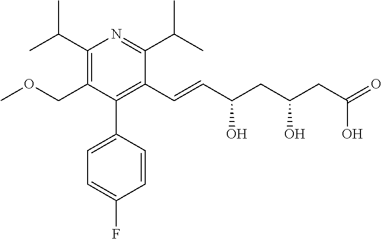 |
| Compactin | (2S)-2-methyl-butanoic acid (1S,7S,8S,8aR)- 1,2,3,7,8,8a-hexahydro- 7-methy1-8-[2- [(2R,4R)-tetrahydro-4- hydroxy-6-oxo-2H- pyran-2-yflethy1]-1- naphthale-nyl ester | 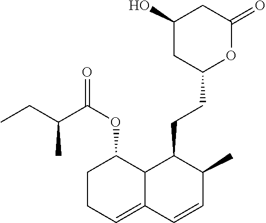 |
| Fluvastatin | (3R,5S,6E)-rel-7-[3-(4- fluorophenyl)-1-(1- methylethyl)-1H-indol- 2-yl]-3,5--dihydroxy-6- heptenoic acid | 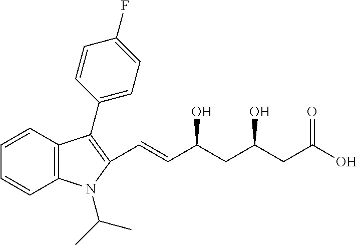 |
| Lovastatin | 2(S)-2-methyl-butanoic acid (1S,3R,7S,8S,8aR)- 1,2,3,7,8,8a-hexahydro- 3,7-dimethyl-8-[2- [(2R,4R)-tetrahydro-4- hydroxy-6-oxo-2H- pyran-2-yl]ethyl]-1-na- phthalenyl ester | 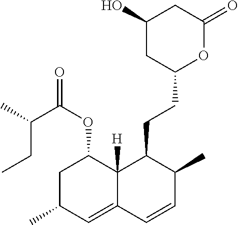 |
| Mevastatin | ((1S,7S,8S,8aR)-8-{2- [(2R,4R)-4-hydroxy-6- oxotetrahydro-2H- pyran-2-yl]ethyl}-7- methyl-1,2,3,7,8,8a- hexahydronaphthalen- 1-yl (2S)-2- methylbutanoate); | 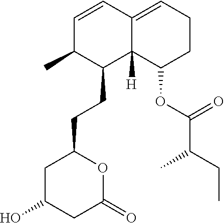 |
| Simvastatin | 2,2-dimethyl-butanoic acid (1S,3R,7S,8S,8aR)- 1,2,3,7,8,8a-hexahydro- 3,7-dimethyl-8-[2- [(2R,4R)-tetrahydro-4- hydroxy-6-oxo-2H- pyran-2-yl]ethyl]-1- naphthalenyl ester | 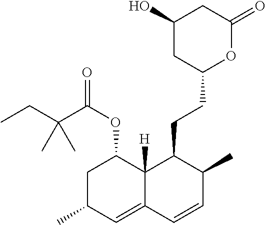 |
| Pitavastatin | (3R,5S,6E)-742- cyclopropyl-4-(4- fluorophenyl)quinolin- 3-yl]-3,5- dihydroxyhept-6-enoic acid | 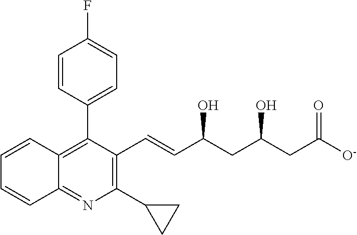 |
| Pravastatin | (βR,δR,1 S,2S,6S,8S,8aR)- 1,2,6,7,8,8a-hexahydro- β,δ,- 6-trihydroxy-2- methyl-8-[(2S)-2- methyl-1-oxobutoxyl- 1-naphthaleneheptanoic acid | 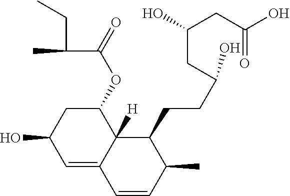 |
| Rosuvastatin | (3R,5S,6E)-7-[4-(4- fluorophenyl)-6-(1- methylethyl 2-[methyl (methylsulfonyl-) amino]-5-pyrimidinyl]- 3,5-dihydroxy-6- heptenoic acid | 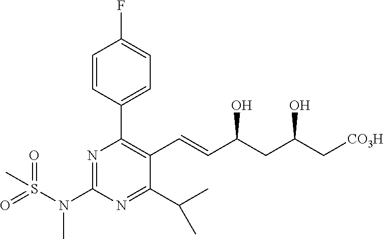 |

Other suitable HMG CoA reductase inhibitors are taught in U.S. Patent Application Publication No. 2005/0239871, which is herein incorporated by reference in its entirety.

In an embodiment of the present invention, methods for interfering with the activity of CTGF or treating cancer metastasis comprise administration of compounds or compositions comprising at least one HMG-CoA reductase inhibitor. In an exemplary embodiment of the present invention, the at least one HMG-CoA reductase inhibitor is simvastatin

The pharmaceutically acceptable salts and solvates, and prodrug forms of CTGF inhibitory compounds and compositions described herein can also be used in the compositions and methods of the present invention. Furthermore, derivatives of the compounds taught herein can also be used in the methods of the present invention. Derivatives include, but are not limited to: derivatives of carboxylic acids (for example: carboxylic acid salts, esters, lactones, amides, hydroxamic acids, alcohols, esterified alcohols and alkylated alcohols (alkoxides)) and derivatives of alcohols (for example: esters, carbamates, lactones, carbonates, alkoxides, acetals, ketals, phosphates, and phosphate esters). Where a fluorine is found on one or more of the aromatic rings, any other halide can be used. Also, in lieu of hydrogen or alkyl groups, different alkyl groups can be used. For instance, instead of an —OH group, an —O-alkyl group could be used. Thus, various derivatives can easily be used in the present invention based on the guidance and knowledge presented herein, together with the knowledge that one skilled in the art has in this technical area. It is recognized that the compounds can contain one or more chiral centers. This invention contemplates all enantiomers, diastereomers, and mixtures thereof.

In an embodiment of the present invention, methods for interfering with the activity of CTGF or treating cancer metastasis comprise administration of compounds or compositions further comprising at least one HMG-CoA reductase inhibitor and an active agent. As used herein, the term “active agent” can include, without limitation, agents for gene therapy, analgesics, antiarthritics, antiasthmatic agents, anticholinergics, anticonvulsants, antidepressants, antidiabetic agents, antidiarrheals, anesthetics, antibiotics, antigens, antihistamines, anti-infectives, anti-inflammatory agents, antimicrobial agents, antimigraine preparations, antinauseants, antineoplastics, antiparkinsonism drugs, antipruritics, antipsychotics, antipyretics, antispasmodics, anorexics, antihelminthics, antiviral agents, nucleic acids, DNA, RNA, polynucleotides, nucleosides, nucleotides, amino acids, peptides, proteins, carbohydrates, lectins, lipids, fats, fatty acids, viruses, antigens, immunogens, antibodies and fragments thereof, sera, immune stimulants, immune suppressors, sympathomimetics, xanthine derivatives, cardiovascular agents, potassium channel blockers, calcium channel blockers, beta-blockers, alpha-blockers, antiarrhythmics, antihypertensives, diuretics, antidiuretics, vasodilators comprising general, coronary, peripheral, or cerebral, central nervous system stimulants, vasoconstrictors, gases, growth factors, growth inhibitors, hormones, estradiol, steroids, progesterone and derivatives thereof, testosterone and derivatives thereof, corticosteroids, angiogenic agents, antiangeogenic agents, hypnotics, immunosuppressives, muscle relaxants, parasympatholytics, psychostimulants, sedatives, tranquilizers, ionized and non-ionized active agents, anti-fungal agents, metals, small molecules, pharmaceuticals, hemotherapeutic agents, wound healing agents, indicators of change in the bio-environment, enzymes, nutrients, vitamins, minerals, coagulation factors, neurochemicals, cellular receptors, radioactive materials, cells, chemical or biological materials or compounds that induce a desired biological or pharmacological effect; and combinations thereof.

In an exemplary embodiment of the present invention, an active agent can be Vitamin D and derivatives thereof including ergocalciferol, cholecalciferol, 22-dihydroergocalciferol, sitocalciferol, or combinations thereof, among others. In another exemplary embodiment of the present invention, an active agent can be Vitamin C (i.e., asorbic acid) or derivatives thereof. In yet another exemplary embodiment of the present invention, an active agent can be capsaicin or derivatives thereof. In yet another exemplary embodiment of the present invention, an active agent can be an inhibitor of cathepsins. In still another exemplary embodiment of the present invention, an active agent can be a caspase.

Methods and compositions of the present invention comprise administering an effective amount of a compound or composition to treat, ameliorate or prevent disorders resulting from CTGF activity in a subject. The compounds or compositions of the presence invention comprise inhibitors of CTGF expression or CTGF activity. A method of treating a disorder in a subject may comprise administering an effective amount of a CTGF inhibiting compound or composition to a subject with a disorder due to CTGF activity. Such compounds or compositions may comprise an antibody or fragment thereof capable of binding at least a portion of CTGF, a small molecule (e.g., an HMG CoA reductase inhibitor), an interfering peptide, an interfering nucleic acid (e.g., siRNA, antisense RNA) that interferes with the activity of CTGF or a cellular factor related thereto, interferes with the transcription of CTGF genes or genes related to the transcription of CTGF genes, or interfere with the translation of CTGF RNA or the RNAs of cellular factors that affect the expression or activity of CTGF.

Methods and compositions of the present invention comprise treatment of a subject having neoplastic disease. Methods and compositions of the present invention comprise administering an effective amount of a compound or composition to treat, ameliorate or prevent neoplastic diseases in a subject. More specifically, methods and compositions of the present invention comprise treatment of a human having neoplastic disease. Neoplastic disease may occur in any organ or tissue including, but not limited to, bone, brain breast, cervix, colon, endometrium, esophagus, eye, gallbladder, kidney, liver, lung, lymphoid, mucosal, neuronal, ovary, pancreas, prostate, rectal, skin, stomach, and/or testicles, among others. The term “neoplastic disease” is intended to refer to cells that have uncontrollable growth, hyperplasia, tumors, tumorigenesis, cancer, metastasis and the like. The methods and compositions of the present invention may be used in combination with other treatments for neoplastic disease know in the art including, but not limited to, surgery, radiation therapy, chemotherapy, immunotherapy, and targeted therapies. In addition, the methods and compositions of the present invention can be utilized for treating a radiation-induced cell growth as well as for the treatment of cancers that demonstrate some resistance to a chemotherapeutic agent.

Such compounds or compositions can comprise an antibody or fragment thereof capable of binding at least a portion of CTGF, a small molecule (e.g., an HMG CoA reductase inhibitor), an interfering peptide, an interfering nucleic acid (e.g., siRNA, antisense RNA) that interferes with the activity of CTGF or a cellular factor related thereto, interferes with the transcription of CTGF genes or genes related to the transcription of CTGF genes, or interfere with the translation of CTGF RNA or the RNAs of cellular factors that affect the expression or activity of CTGF.

In the methods of therapy of the present invention and in the use of compositions according to the invention, a therapeutically effective amount of a CTGF inhibitor compound or composition can be administered to a subject requiring therapy. A “therapeutically effective amount” or “an effective amount” in the context of the present invention is considered to be any quantity of the one or more inhibitor compounds or compositions which, when administered to a subject, causes prevention, reduction, remission, regression, or elimination of a CTGF-related pathology. For example, in the context of cancer, “an effective amount” is considered to be any quantity of the one or more inhibitor compounds or compositions which, when administered to a subject causes prevention, reduction, remission, regression, or elimination of tumorigenesis and/or metastasis, reducing the cell growth of a cancer, reducing the invasiveness of a cancer in a human, or reducing the angiogenesis of a cancer in a human.

The amount of the inhibitor compound or composition, such as a statin or derivative thereof, that can be used in the compositions or methods of the present invention can be determined using in vitro assays and by other methods known to those skilled in the art, such as pre-clinical and clinical trials. Furthermore, tolerable, therapeutically effective amounts of statins for use as anti-cholesterol agents are known and can be obtained from the appropriate supplier or, for example, the U.S. Food and Drug Association (www.fda.gov).

An effective dose of a CTGF-inhibitory compound or composition may be administered daily, more than one time a day, weekly, monthly, or over one or more years to treat or prevent CTGF related pathologies and in treating neoplastic disease, including metastasis. An effective dose may comprise from 0.02 μg to 200 mg/kg subject of an HMG-CoA reductase inhibitor compound, from 0.001 μg to 1,000 mg/kg subject of antibodies that bind to at least a portion of CTGF, from 0.001 μg to 1,000 mg/kg subject of interfering peptides that later the expression or activity of CTGF, or from 0.001 μg to 1,000 mg/kg subject of interfering nucleic acids that later the expression or activity of CTGF.

Compositions of the present invention may be formulated according to protocols well known in the art. Suitable formulations may be determined based on the preferred route by that the inhibitor of CTGF and/or active agent is to be administered. Compositions of the present invention may be prepared in forms suitable for administration by oral dosage forms known in the pharmaceutical arts, including, but not limited to, tablets, capsules, oral liquid formulas, quick dissolve tablets, buccal and other mucosal dosage formulas, inhalation, topical administration, ophthalmic administration, injection, or implantation.

Compositions of the present invention comprise formulations known for administration of therapeutic agents. The compositions may be in the form of a powder, tablet, capsule, liquid, ointment, cream, gel, hydrogel, aerosol, spray, micelle, transdermal patch, liposome, or any other suitable form that may be administered to a subject.

Compositions, such as statins or statin derivatives, administered intranasally or by inhalation can be delivered in the form of a dry powder inhaler or an aerosol spray presentation from a pressurized container, pump, spray or nebulizer with the use of a suitable propellant, carbon dioxide or other suitable gas. In the case of a pressurized aerosol, the dosage unit may be determined by providing a valve to deliver a metered amount. The pressurized container, pump, spray or nebulizer may contain a solution or suspension of the active compound, e.g. using a mixture of ethanol and the propellant as the solvent, which may additionally contain a lubricant (e.g., sorbitan trioleate). Capsules and cartridges (made, for example, from gelatin) for use in an inhaler or insufflator may be formulated to contain a powder mix of a compound of the invention and a suitable powder base such as lactose or starch.

Aerosol or dry powder formulations are provided so that each metered dose contains a suitable quantity of an inhibitor compound or composition, such as statins or statin derivatives, for delivery to the subject. It will be appreciated that the overall daily dose with an aerosol will vary from subject to subject, and may be administered in a single dose or, more usually, in divided doses throughout the day. Nanoparticulated compounds or compositions, such as statins or statin derivatives, may be prepared using techniques known in the art.

Methods of treatment or prevention of CTGF pathologies or neoplastic disease in the eye may require that the inhibitor compound or composition, such as a statin or statin derivative, be prepared as a liquid formulation to the eye. For ophthalmic use, the inhibitor compound or compositions, such as statins or statin derivatives, may be formulated as micronized suspensions in isotonic, pH adjusted, sterile saline, or, preferably, as solutions in isotonic, pH adjusted, sterile saline, optionally in combination with a preservative such as a benzylalkonium chloride.

Methods of treatment or prevention of CTGF pathologies or neoplastic disease in the skin or integumentary system may require that the inhibitor compound or composition, such as a statin or statin derivative, may be prepared for topical administration directly to the skin. For topical application to the skin, the inhibitor compound or compositions, such as statins or statin derivatives, can be formulated as a suitable ointment containing one or more active compounds or compositions suspended or dissolved in, for example, a mixture with one or more of the following: mineral oil, liquid petrolatum, white petrolatum, propylene glycol, polyoxyethylene polyoxypropylene compound, emulsifying wax, and water. Alternatively, formulations may be a lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol, and water.

Liquid vehicles can be used in preparing solutions, suspensions, emulsions, syrups, elixirs, and pressurized compositions. The liquid vehicle can contain suitable pharmaceutical additives such as solubilizers, emulsifiers, buffers, preservatives, sweeteners, flavoring agents, suspending agents, thickening agents, colors, viscosity regulators, stabilizers, or osmo-regulators. Suitable examples of liquid vehicles for oral and parenteral administration include, but are not limited to, water (partially containing additives as above, e.g. cellulose derivatives, such as a sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the vehicle can be an oily ester, such as ethyl oleate and isopropyl myristate. Sterile liquid vehicles are useful in sterile liquid form compositions for parenteral administration. The liquid vehicle for pressurized compositions can be halogenated hydrocarbons or other pharmaceutically acceptable propellant.

Liquid pharmaceutical compositions, which are sterile solutions or suspensions can be utilized by, for example, intramuscular, intrathecal, intratumoral, epidural, intraperitoneal, or subcutaneous injection. Sterile solutions can also be administered intravenously. The inhibitory compounds or compositions may be prepared as a sterile solid composition, which may be dissolved or suspended at the time of administration using sterile water, saline, or other appropriate sterile injectable medium. Vehicles are intended to include necessary and inert binders, suspending agents, lubricants, flavorants, sweeteners, preservatives, dyes, and coatings.

Optimal dosages to be administered to a subject for the interfering with the activity of CTGF or treating a CTGF-related disorder, such neoplastic disease, may be determined by those skilled in the art, and will vary with the particular disease, subject, and treatment or prevention protocol in use, the strength of the preparation, the mode of administration, and the advancement of the disease condition that is to be treated. Additional factors depending on the particular subject being treated will result in a need to adjust dosages, including subject age, weight, gender, diet, and time of administration.

Known procedures, such as those conventionally employed by the pharmaceutical industry (e.g. in vivo experimentation, clinical trials etc), may be used to establish specific formulations of compositions and precise therapeutic regimes (such as daily doses of the inhibitory compounds and compositions, and the frequency of administration). Daily doses may be given as a single administration (e.g. a daily tablet for oral consumption or as a single daily injection). Alternatively, the inhibitory compounds and compositions used may require administration two or more times during a day, dependent on pharmacological, toxicological or efficacy studies.

Another aspect of the present invention comprises systems and methods for diagnosing or determining the progression of cancer in a subject. Systems and methods for diagnosing or determining the progression of cancer in a subject comprise a probe capable of detecting the expression of connective tissue growth factor (CTGF) in a subject. The systems and methods for diagnosing or determining the progression of cancer in a subject can permit discrimination between metastatic and non-metastatic cancer. The systems and methods of the present invention can comprise a probe for at least one cellular marker for metastasis. The at least one cellular marker for metastasis can include, but is not limited to CTGF, E-cadherin, N-cadherin, vimentin, fibronectin, α-smooth muscle actin (SMA), or other markers for the epithelial-to mesenchymal transition (EMT). As used herein, the term “probe” can refer to an antibody or fragment thereof capable of binding at least a portion of at least one cellular marker for metastasis, a nucleic acid (e.g., DNA or RNA) capable of binding at least a portion of the DNA or RNA encoding at least one cellular marker for metastasis, among others. Therefore, the systems and methods of the present invention are capable of detecting protein or gene expression of at least one cellular marker for metastasis. In an exemplary embodiment of the present invention, the systems and methods of the present invention can detect expression of CTGF protein. In another exemplary embodiment of the present invention, the systems and methods of the present invention can detect expression of CTGF mRNA.

The systems and methods of the present invention are capable of detecting protein or gene expression of at least one cellular marker for metastasis in a subject. The systems and methods of the present invention are capable of detecting protein or gene expression of at least one cellular marker for metastasis in a subject a tumor cell or a mesenchymal cell or a plurality of tumor cells or mesenchymal cells. As used herein, the term “mesenchymal cell” includes, but is not limited to, fibroblasts, myofibroblasts, endothelial cells, and smooth muscle cells.

The systems and methods for diagnosing or determining the progression of cancer in a subject can assay the expression of at least one cellular marker for metastasis in a sample derived from a subject relative to a control sample. Based on expression levels (e.g., RNA or protein), a determination can be made whether the sample derived from a subject is metastatic. For example, in metastatic cells, expression of CTGF, N-cadherin, vimentin, fibronectin and α-SMA is up-regulated, whereas expression of E-cadherin is down-regulated as compared to non-metastatic cells. Thus, the systems and methods of the present invention can comprise a positive and negative control for expression of at least one cellular marker for metastasis. In such an embodiment, the expression profile for at least one cellular marker for metastasis in the sample derived from the subject can be compared to the expression profile for at least one cellular marker for metastasis in the negative and/or positive control. A sample may be derived from a bodily fluid of a subject (e.g., blood) or from a biopsy of a tumour from a subject, among others.

It must be noted that, as used in this specification and the appended claims, the singular forms “a,” “an,” and “the” include plural referents unless the context clearly dictates otherwise. In addition, all patents, patent applications and references included herein are specifically incorporated by reference in their entireties.

It should be understood, of course, that the foregoing relates only to exemplary embodiments of the present invention and that numerous modifications or alterations may be made therein without departing from the spirit and the scope of the invention as set forth in this disclosure. Although the exemplary embodiments of the present invention are provided herein, the present invention is not limited to these embodiments. There are numerous modifications or alterations that may suggest themselves to those skilled in the art.

The present invention is further illustrated by way of the examples contained herein, which are provided for clarity of understanding. The exemplary embodiments should not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

Therefore, while embodiments of this invention have been described in detail with particular reference to exemplary embodiments, those skilled in the art will understand that variations and modifications can be effected within the scope of the invention as defined in the appended claims. Accordingly, the scope of the various embodiments of the present invention should not be limited to the above discussed embodiments, and should only be defined by the following claims and all equivalents.

### EXAMPLES

### Example 1

### Elevated Expression of CTGF During Prostate Cancer Progression

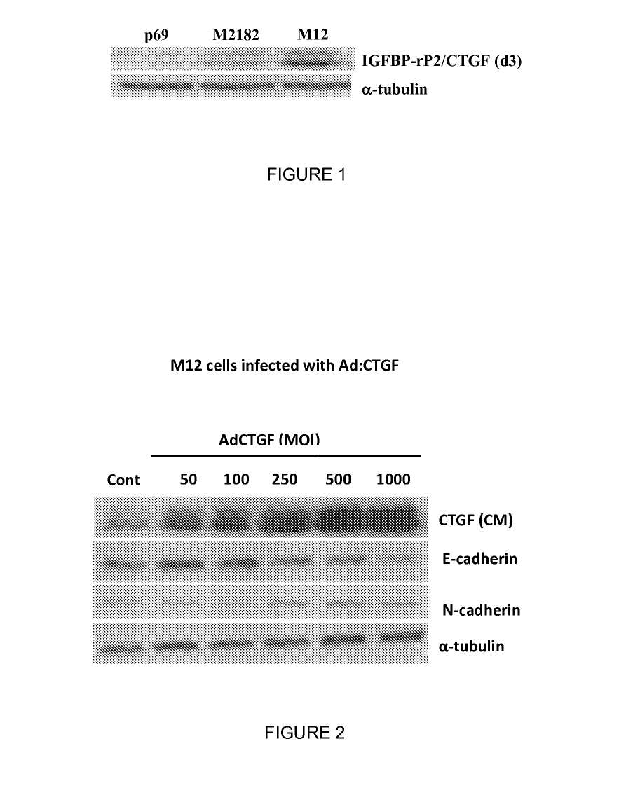

Simian virus-40 (SV40)-T antigen-immortalized prostate epithelial cells, P69SV40-T (P69) and two sublines generated from the parental line by serial passage through athymic mice, one tumorigenic (M2182) and one metastatic (M12), were grown in serum-free media for three days. Conditioned media (CM) were collected and subjected to Western immunoblotting after adjustment of total protein concentrations. As demonstrated by the data presented in FIG. 1, CTGF expression is highly elevated in tumorigenic and metastatic M12 prostate cancer cells but not in normal p69 cells or non-metastatic M2182 prostate cancer cells.

### Example 2

### Biological Significance of IGFBP-RP2/CTGF in Prostate Cancer Cells

M12 cells were infected with an adenoviral vector expressing CTGF (AdCTGF) at various multiplicities of infection (MOI) ranging from 50-1000, and the infection was allowed to occur for two days. (FIG. 2). Conditioned media (CM) and cell lysates (CL) were collected and subjected to Western immunoblot analysis after adjustment of total protein concentrations.

As indicated in FIG. 2, over-expression of CTGF resulted in induction of specific EMT markers, such as α-smooth muscle actin (α-SMA) and fibronectin (FN) in M12 cells. These data demonstrate that overexpression of CTGF using either retroviral vector or adenoviral vector expressing CTGF resulted in a significant change of EMT markers, such as induction of α-SMA, fibronectin, and N-cadherin and suppression of E-cadherin in M12 metastatic prostate cancer cells. This is the very first experimental evidence showing the connection between CTGF and EMT induction. Since EMT appears to be a critical event for local invasion and metastasis of many cancer, our data strongly suggest that CTGF promotes prostate cancer cell invasion and metastasis via induction of EMT, as cooperating with, or being a downstream mediator of these EMT-inducing growth factors.

### Example 3

### Impact of Statins on the Expression and Biological Function of CTGF in Prostate Cancer Cells and Breast Cancer Cells

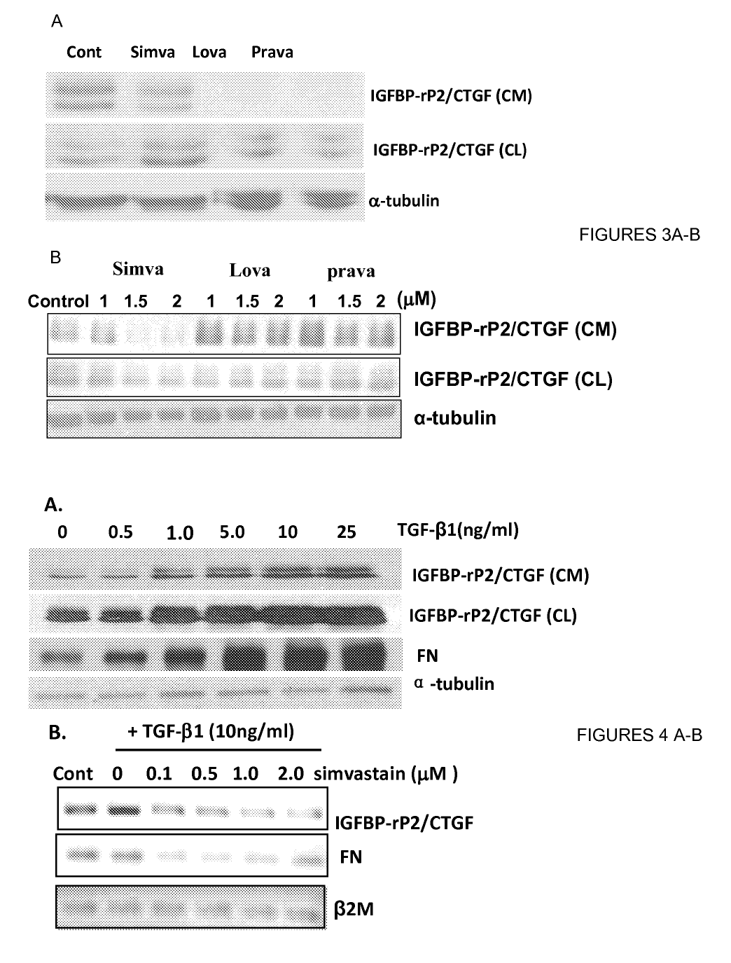

The effect of statins on the TGF-β- and CTGF-induced EMT in prostate cancer cells was further tested. Several stains are available with different pharmacokinetics and biotransformation rates. In particular, statins have different solubilities—some are more fat soluble (lovastatin, simvastatin, atorvastatin, and fluvastatin) and some are more water soluble (rosuvastatin, mevastatin, and pravastatin)). The hydrophilic statins are absorbed preferentially by the liver, where they inhibit production of cholesterol. More lipophilic statins are readily distributed to sites beyond the liver, where they inhibit intracellular synthesis of cholesterol. These different pharmacokinetic properties of statins may greatly influence effectiveness against advanced prostate cancer in vivo. For this reason, two lipophilic statins (lovastatin and simvastatin) and a hydrophilic statin (pravastatin) were selected for initial in vitro studies. FIG. 3 shows that treatment of 1 μM simvastatin, lovastatin and pravastatin for four days resulted in significant suppression of CTGF in M12 prostate cancer cells (FIG. 3A) and MDA 231 breast cancer cells (FIG. 3B). This data clearly demonstrates that statins inhibit endogenous CTGF of which expression is elevated in metastatic M12 prostate cancer cells and MDA231 breast cancer cells.

### Example 4

### Impact of Statins on the Expression and Biological Function of IGFBP-RP2/CTGF in Prostate Stroma

Previous studies have demonstrated the elevated expression of CTGF as well as its induction by TGF-β in prostate stroma. Furthermore, when mouse prostate stromal fibroblasts, expressing retroviral-introduced CTGF, were combined with LNCaP human prostate cancer cells in the DRS xenograft tumor model, stromal expression of CTGF resulted in an increase of angiogenesis and tumorigenesis of prostate cancer. These studies strongly suggest that stromal expression of IGFBP-rP2/CTGF by TGF-β and other growth factors may influence prostate cancer cell invasion and metastasis through autocrine and/or paracrine fashion.

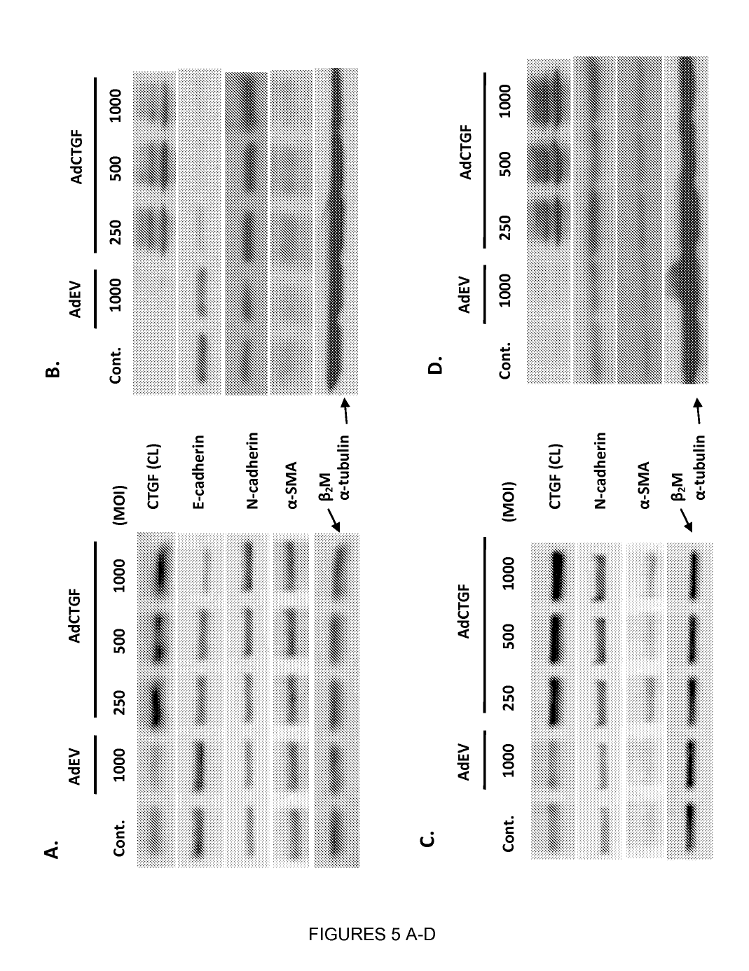

In order to investigate the effect of statins on endogenous as well as TGF-β-induced CTGF in prostate stroma, normal immortalized human lung fibroblasts were used to characterize the effect of TGF-β1 and statins on stromal expression of CTGF. When stromal cells were treated with TGF-β1 ranging from 0 ng/ml to 25 ng/ml for 3 days, the expression of CTGF and fibronectin were significantly increased in a concentration dependent manner as shown in the Western immunoblot of FIG. 4A.

As shown in FIG. 4A, dosage of TGF-β1 at a concentration of 10 ng/ml elicited a robust production of CTGF. In order to assess the effects of statins on basal CTGF and TGF-β1-induced CTGF, stromal fibroblasts were treated with 10 ng/ml TGF-β1 in the presence of simvastatin, ranging from concentrations of 0.1 μM to 2.0 μM for 20 hours. Total RNAs were extracted and were subjected to semi-quantitative RT-PCR. As shown in FIG. 4B, statin treatment resulted in suppression of basal as well as TGF-β-induced CTGF in a dose dependent manner in stromal fibroblasts (FIG. 4B). Furthermore, TGF-β-induced expression of CTGF and fibronectin was abrogated by simvastatin treatment. Taken together, it is evident that statins suppress CTGF expression in prostate cancer cells as well as cancer-associated reactive stroma, thereby inhibiting CTGF-induced EMT and subsequent invasion and metastasis in prostate cancer.

### Example 5

### Impact of Statins on the IGFBP-RP2/CTGF-Induced EMT Markers in Prostate and Breast Cancer Cells

In order to identify direct effect of CTGF on EMT induction in human cancer, an adenoviral expression vector was used. M12 (FIGS. 5 A-B) and M2182 (FIGS. 5 C-D) prostate cancer cells were infected with either an empty adenoviral constructs or an adenoviral vector expressing CTGF (AdCTGF) for either 18 hrs or 2 days to collect total RNAs for RT-PCR (FIGS. 5 A and C) and proteins for Western immunoblot analysis (FIGS. 5 B and D), respectively. As shown in FIG. 5, adenoviral-transduced CTGF resulted in a significant increase of mesenchymal marker expression, such as N-cadherin and α-SMA, and a significant decrease of the epithelial marker, E-cadherin, at the mRNA level and protein level in M12 cells. (FIGS. 5 A-B). Similar to the results observed in M12 cells, adenoviral-transduced CTGF resulted in a significant increase of mesenchymal marker expression, such as N-cadherin and α-SMA at the mRNA level and protein level in M2182 cells (FIGS. 5 C-D); however, E-cadherin was undetectable in M2182 prostate cancer cells. The control adenovirus (AdEV) showed no changes in EMT marker expression.

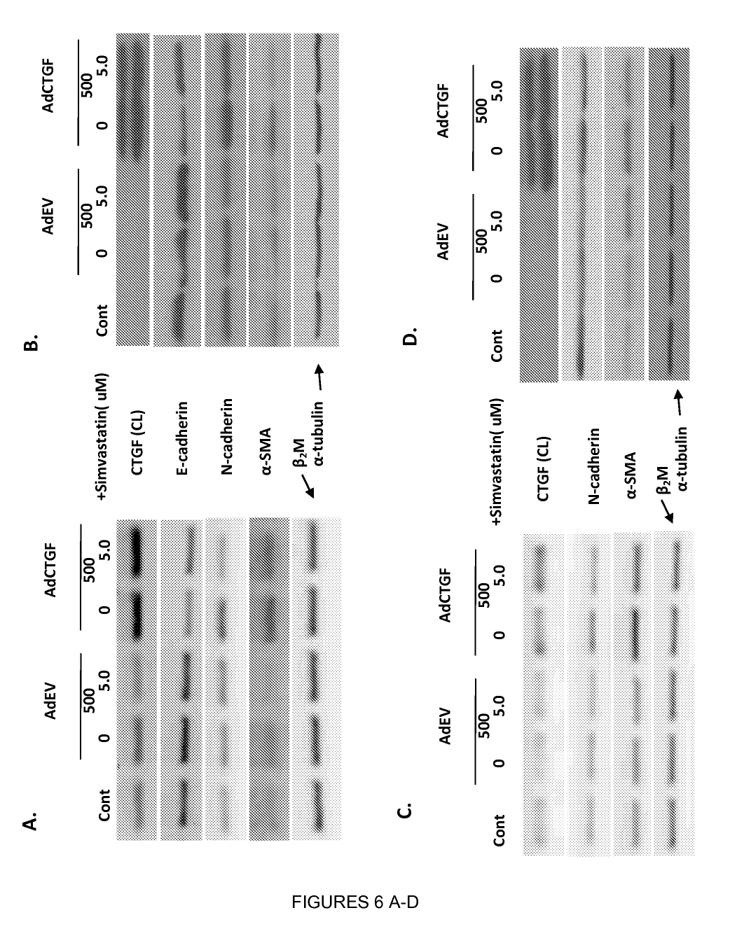

The effects of statins on CTGF-induced EMT were investigated using 5 μM simvastatin. M12 (FIGS. 6 A-B) and M2182 (FIGS. 6 C-D) prostate cancer cells were infected with either an empty adenoviral construct (ADEV) or an adenoviral vector expressing CTGF (AdCTGF) for either 18 hrs or 2 days to collect total RNAs for RT-PCR (FIGS. 5 A and C) or proteins for Western immunoblot analysis (FIGS. 5 B and D), respectively. As shown in FIG. 6, simvastatin treatment efficiently suppressed the CTGF-induced increase of N-cadherin and α-SMA, and simvastatin treatment resulted in an increase of the epithelial marker E-cadherin at mRNA level and protein level in M12 cells (FIGS. 6 A-B). Similar to the resulted observed in M12 cells, simvastatin treatment efficiently suppressed the CTGF-induced increase of N-cadherin and α-SMA in M2182 cells at the mRNA and protein level (FIGS. 6 C-D). Simvastatin (e.g., at concentrations of 11 μM, 2 μM, 4 μM, 5 μM), however, did not affect adenoviral transduced IGFBP-rP2/CTGF expression, strongly suggesting that simvastatin inhibits CTGF signaling pathways involved in expression of EMT markers.

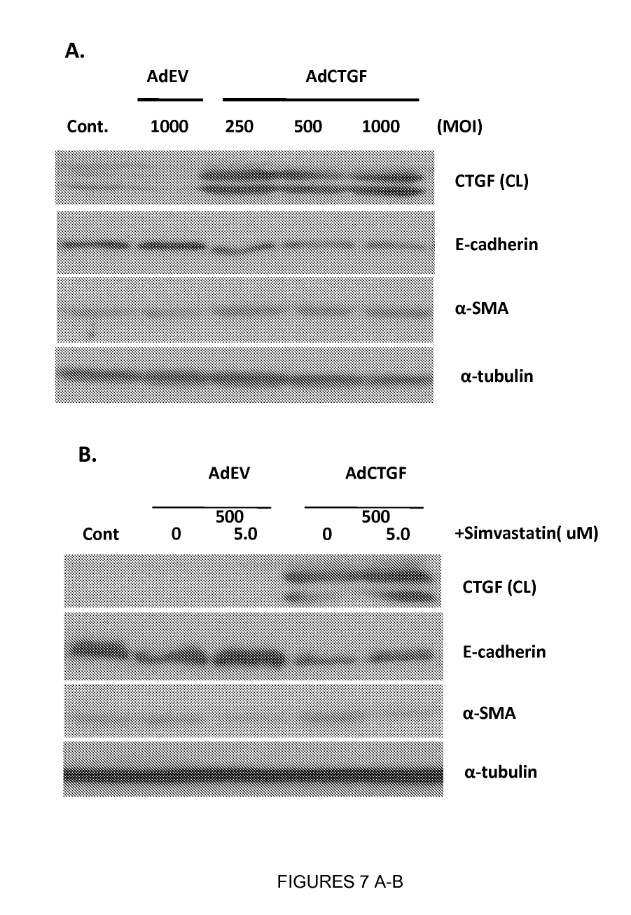

Similar effects of simvastatin on CTGF-induced EMT induction were observed in breast cancer cells. As shown in FIG. 7, MCF-7 breast cancer cells were infected with either empty adenoviral construct (AdEV) or an adenoviral vector expressing CTGF (AdCTGF) in the presence or absence of 5 μM simvastatin for 2 days. Cell lysates (CL) were collected and subjected to Western immunoblot analysis after adjustment of total protein concentrations. Overexpression of CTGF resulted in an increase of mesenchymal marker α-SMA expression, whereas a significant decrease in expression of epithelial marker E-cadherin was observed in MCF-7 breast cancer cells.

Collectively, these findings indicate that CTGF-induced EMT and metastasis can be inhibited by statins in a variety of human cancers. The ability of CTGF to induce EMT and cell proliferation is of much relevance for cancer cell growth, invasion, and metastasis. Thus, these data demonstrate that CTGF is not only an excellent marker for cancer progression and prognosis but also molecular target for metastasis. Furthermore, the specific inhibitory actions of statins on CTGF provide a strong rationale for the use of statins in the prevention and treatment of advanced cancer.

### Example 6

### Induction of CTGF and Fibronectin with Radiation in Prostate and Lung Cancer Cells

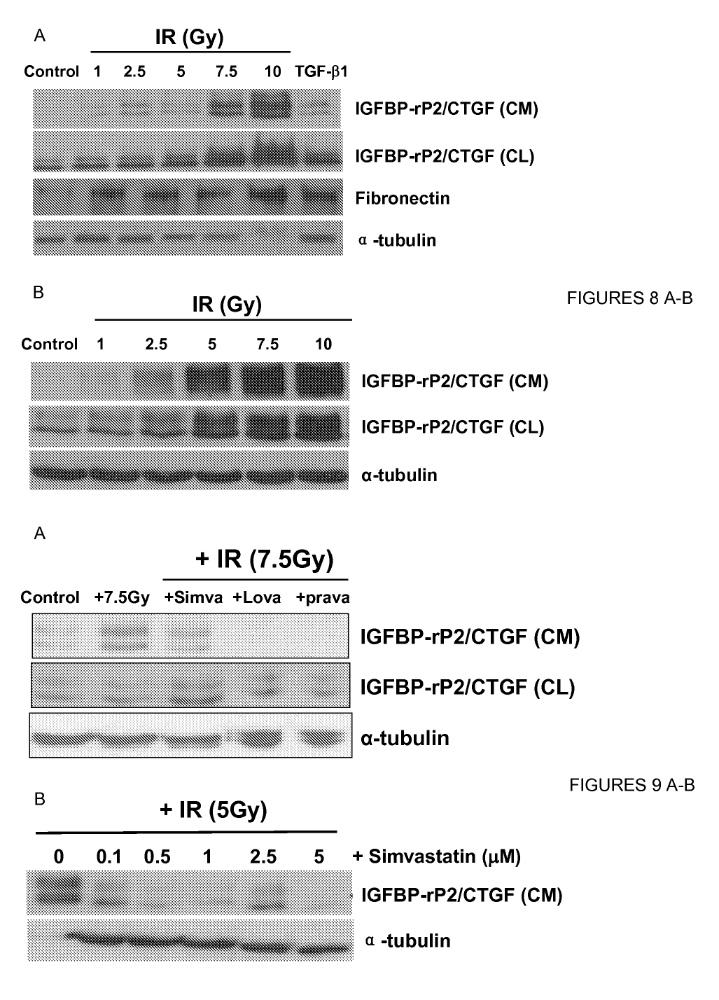

In an effort to determine the mechanism of radiation-induced adverse effects, such as radio-resistance and increased potential of metastasis in human cancer, M12 prostate cancer cells and BEAS-2B-CSC lung cancer cells were exposed to increasing amounts of radiation, ranging from 0 to 10 Grays (Gy). To detect protein expression for CTGF and CTGF-regulated protein fibronectin, M12 and BEAS-2B-CSC cells were harvested 3 days after irradiation, and culture media (CM) and cell lysates (CL) were separated by SDS-PAGE. Western immunoblotting was performed with antibodies specific for CTGF, fibronectin, and α-tubulin. As indicated in FIG. 8, the amount of CTGF in both the culture media (CM) and the cell lysates (CL) increased as the amount of radiation increased in M12 prostate cancer cells (FIG. 8A) and BEAS-2B-CSC lung cancer cells (FIG. 8B). As a control for protein loading, α-tubulin expression was also determined.

### Example 7

### Inhibitory Effect of Statins on Radiation—Induction of CTGF

The ability of statins to inhibit radiation-induced CTGF expression was assessed in M12 prostate cancer cells and BEAS-2B-CSC lung cancer cells. FIG. 9A shows that treatment with 1 μM simvastatin, lovastatin, or pravastatin for four days resulted in a significant suppression of radiation-induced CTGF expression in M12 prostate cancer cells. Similar to the results of statin treatment in FIG. 9A, BEAS-2B-CSC lung cancer cells show a dose-dependent inhibitory effect of simvastatin on radiation-induction of CTGF (FIG. 9B). These data clearly demonstrate that statins inhibit radiation-induced CTGF, which plays a critical role cell proliferation, angiogenesis, and metastasis. Collectively, these findings indicate that radiation-induction of CTGF can induce Epithelial-mesenchymal transition and metastasis as well as proliferation, angiogenesis, invasion and resistance, and statins can inhibit these adverse effects of radiotherapy in a variety of human cancer.

What is claimed is:

1. A method for interfering with the activity of connective tissue growth factor (CTGF), comprising, administering to a subject an effective amount of a composition comprising an inhibitor of CTGF.

2. The method of claim 1, wherein the composition prevents the transcription of CTGF genes or translation of CTGF mRNA.

3. The method of claim 1, wherein the composition interferes with the activity of CTGF cellular signaling pathways.

4. The method of claim 2, wherein the composition that interferes with the activity comprises an antibody or a fragment thereof that binds to at least a portion of CTGF, a peptide, a nucleic acid, or small molecule.

5. The method of any of the preceding claims, wherein the composition comprises at least one HMG-CoA reductase inhibitor compound.

6. The method of claim 5, wherein at least one HMG-CoA reductase inhibitor compound is selected from atorvastatin, berivastatin, cerivastatin, compactin, dalvastatin, dihydromevinolin, fluvastatin, glenvastatin, lovastatin, mevastatin, nisvastatin, pravastatin, pitavastatin, rivastatin, rosuvastatin, simvastatin, visastatin, or combinations thereof.

7. The method of claim 5, wherein the HMG-CoA reductase inhibitor compound is simvastatin.

8. The method of any of the preceding claims, wherein the composition further comprises at least one of Vitamin D or derivatives thereof, Vitamin C or derivatives thereof, capsaicin or derivatives thereof, inhibitors of cathepsins, caspases, or combinations thereof.

9. The method of claim 1, wherein the activity of CTGF is associated with at least one of cancer metastasis, cancer cell growth, cancer invasion, and cancer angiogenesis.

10. The method of claim 9, wherein the activity of CTGF is associated with prostate cancer metastasis.

11. The method of claim 9, wherein the activity of CTGF is associated with breast cancer metastasis.

12. A pharmaceutical composition comprising an inhibitor of connective tissue growth factor (CTGF).

13. The composition of claim 12, wherein the inhibitor of CTGF compromises an antibody or a fragment thereof that binds to at least a portion of CTGF, a peptide, a nucleic acid, or small molecule and is capable of at least one of interfering with the activity of CTGF; preventing the transcription of CTGF genes; or translation of CTGF mRNA.

14. The composition of claim 12, wherein the inhibitor of CTGF comprises at least one HMG-CoA reductase inhibitor compound.

15. The composition of claim 14, wherein at least one HMG-CoA reductase inhibitor compound is selected from atorvastatin, berivastatin, cerivastatin, compactin, dalvastatin, dihydromevinolin, fluvastatin, glenvastatin, lovastatin, mevastatin, nisvastatin, pravastatin, pitavastatin, rivastatin, rosuvastatin, simvastatin, visastatin or combinations thereof.

16. The composition of any of claims 12-15, further comprising at least one of Vitamin D or derivatives thereof, Vitamin C or derivatives thereof, capsaicin or derivatives thereof, inhibitors of cathepsins, caspases, or combinations thereof.

17. A system for diagnosing the progression of cancer in a subject, comprising a probe capable of detecting the expression of connective tissue growth factor (CTGF) in a subject.

18. The system of claim 17, wherein detecting the expression of CTGF in a subject comprises detecting expression of CTGF mRNA in a subject.

19. The system of claim 18, wherein detecting the expression of CTGF mRNA in a subject comprises detecting expression of CTGF mRNA in a tumor cell or a mesenchymal cell.

20. The system of claim 17, wherein detecting the expression of CTGF in a subject comprises detecting expression of CTGF protein in a subject.

21. The system of claim 20, wherein detecting the expression of CTGF protein in a subject comprises detecting expression of CTGF protein in a tumor cell or a mesenchymal cell.

22. The system of claim 17, wherein the probe comprises a nucleic acid or a protein.

23. The system of claim 22, further comprising a detector capable of detecting the interaction of the probe with a target associated with CTGF expression.

24. The system of claim 23, wherein CTGF is expressed in non-metastatic cells at a first amount and CTGF is expressed in metastatic cells at a second amount that is greater than the first amount.

25. A method for treating a neoplastic disease, comprising, administering to a human an effective amount of a composition comprising a statin that inhibits production or activity of connective tissue growth factor (CTGF).

26. The method of claim 25, wherein the statin is selected from one or more of atorvastatin, berivastatin, cerivastatin, compactin, dalvastatin, dihydromevinolin, fluvastatin, glenvastatin, lovastatin, mevastatin, nisvastatin, pravastatin, pitavastatin, rivastatin, rosuvastatin, simvastatin, visastatin, or combinations thereof.

27. The method of claim 25, wherein the statin is simvastatin.

28. The method of claim 25, wherein the composition further comprises at least one of Vitamin D or derivatives thereof, Vitamin C or derivatives thereof, capsaicin or derivatives thereof, inhibitors of cathepsins, caspases, or combinations thereof.

29. The method of claim 25, further comprising at least one of reducing the metastasis of a cancer in a human, reducing the cell growth of a cancer, reducing the invasiveness of a cancer in a human, or reducing the angiogenesis of a cancer in a human.

30. The method of claim 29, wherein the cancer is prostate cancer.

31. The method of claim 29, wherein the cancer is breast cancer.

32. A method for diagnosing the progression of cancer in a subject, comprising:
detecting the expression of connective tissue growth factor (CTGF) in a subject; and
diagnosing the progression of cancer in a subject.

33. The method of claim 32, wherein detecting the expression of CTGF in a subject comprises detecting expression of CTGF mRNA in a subject.

34. The method of claim 32, wherein detecting the expression of CTGF mRNA in a subject comprises detecting expression of CTGF mRNA in a tumor cell or a mesenchymal cell.

35. The method of claim 32, wherein detecting the expression of CTGF in a subject comprises detecting expression of CTGF protein in a subject.

36. The method of claim 32, wherein detecting the expression of CTGF protein in a subject comprises detecting expression of CTGF protein in a tumor cell or a mesenchymal cell.

37. The method of claim 32, wherein diagnosing the progression of cancer in a subject comprises determining the expression of CTGF expression in a subject, wherein CTGF is expressed in non-metastatic state at a first amount and CTGF is expressed in a metastatic state at a second amount that is greater than the first amount.

38. A method for treating a radiation-induced cell growth, comprising, administering to a human an effective amount of a composition comprising a statin that inhibits production or activity of connective tissue growth factor (CTGF).

39. The method of claim 38, wherein the statin is selected from one or more of atorvastatin, berivastatin, cerivastatin, compactin, dalvastatin, dihydromevinolin, fluvastatin, glenvastatin, lovastatin, mevastatin, nisvastatin, pravastatin, pitavastatin, rivastatin, rosuvastatin, simvastatin, visastatin, or combinations thereof.

40. The method of claim 39, wherein the statin is simvastatin.

41. The method of claim 38, wherein the composition further comprises at least one of Vitamin D or derivatives thereof, Vitamin C or derivatives thereof, capsaicin or derivatives thereof, inhibitors of cathepsins, caspases, or combinations thereof.

42. The method of claim 38, further comprising at least one of reducing the metastasis of a cancer in a human, reducing the invasiveness of a cancer in a human, or reducing the angiogenesis of a cancer in a human.

43. The method of claim 42, wherein the cancer is prostate cancer.

44. The method of claim 42, wherein the cancer is breast cancer.

45. The method of claim 42, wherein the cancer is lung cancer.

46. A method for treating a neoplastic disease that demonstrates some resistance to a chemotherapeutic agent, comprising, administering to a human an effective amount of a composition comprising a statin that inhibits production or activity of connective tissue growth factor (CTGF).

47. The method of claim 46, wherein the statin is selected from one or more of atorvastatin, berivastatin, cerivastatin, compactin, dalvastatin, dihydromevinolin, fluvastatin, glenvastatin, lovastatin, mevastatin, nisvastatin, pravastatin, pitavastatin, rivastatin, rosuvastatin, simvastatin, visastatin, or combinations thereof.

48. The method of claim 47, wherein the statin is simvastatin.

50. The method of claim 46, wherein the composition further comprises at least one of Vitamin D or derivatives thereof, Vitamin C or derivatives thereof, capsaicin or derivatives thereof, inhibitors of cathepsins, caspases, or combinations thereof.

51. The method of claim 46, further comprising increasing susceptibility of a cancer to a chemotherapeutic agent.

52. The method of claim 51, wherein the cancer is prostate cancer.

53. The method of claim 51, wherein the cancer is breast cancer.

54. The method of claim 51, wherein the cancer is lung cancer.
